(19) United States
(12) Patent Application Publication
(10) Pub. No.: US 2005/0143347 A1
(43) Pub. Date: Jun. 30, 2005

(54) Medicinal lipolysis of accumulations of fat

(75) Inventors: Peter Boderke, Frankfurt (DE); Matthias Gossel, Hofheim (DE); Walter Kamm, Idstein (DE); Karl-Heinz Nietsch, Neuss (DE); Rainer Pooth, Dreieich-Goetzenhain (DE); Juergen Sandow, Dreieich-Goetzenhain (DE); Joerg Hager, Koeln (DE); Gerhard Sattler, Darmstadt (DE)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma Deutschland GmbH, Frankfurt am Main (DE)

(21) Appl. No.: 11/011,333

(22) Filed: Dec. 14, 2004

Related U.S. Application Data
(60) Provisional application No. 60/567,685, filed on May 3, 2004.

(30) Foreign Application Priority Data
Dec. 22, 2003 (DE) 10361067.7

(51) Int. Cl. A61K 31/685; A61K 31/56; A61K 31/525; A61K 31/195; A61K 31/355

(52) U.S. Cl. 514/78; 514/171; 514/458; 514/251; 514/567

(57) ABSTRACT

Aqueous preparations comprising at least one phospholipid or at least one bile acid and a component assisting degradation of fat such as riboflavin and water are suitable for producing medicaments for removing subcutaneous accumulations of fat and lead to regression of diet-resistant fat pads.

### CROSS-REFERENCE TO RELATED APPLICATIONS

This application claims the benefit of U.S. Provisional Application No. 60/567,685, filed May 3, 2004, and incorporated herein by reference.

### DESCRIPTION OF THE INVENTION

The invention relates to aqueous preparations comprising at least one phospholipid and/or at least one bile acid and a lipophilic compound such as riboflavin and water, which are suitable for producing medicaments for removing subcutaneous accumulations of fat and lead to regression of diet-resistant fat pads.

At present, subcutaneous accumulations of fat or proliferations of adipose cells such as lipomas or lipedemas are treated by surgical means through liposuction or direct surgical removal. Treatment measures of these types of are associated with the known complications or risks caused by anesthesia, local reactions and possible infections, and in some circumstances require admission to a hospital ward.

Aqueous preparations comprising at least one phospholipid and/or at least one bile acid are known for various applications. Thus, these systems are employed for example in the cosmetics sector or for manufacturing pharmaceutical products. These systems are in some cases notable for forming spherical vesicles, which are also referred to as liposomes. Said liposomes have a double lipid membrane boundary to the outside and contain an aqueous phase in their interior. Aqueous preparations comprising at least one phospholipid, at least one bile acid and water are described for example in the European patent application EP 0 615 746. A commercially available product is Essentiale® N i.V. (Rote Liste, March 2003), which is an aqueous preparation comprising phospholipids, bile acid, riboflavin, alpha-tocopherol, ethanol and water and is approved for the treatment of, for example, hepatopathies, acute and chronic hepatitis, fatty degeneration of the liver or hepatic necrosis.

It is known that fatty degeneration of the liver involves an excess fat content of the liver parenchyma (deposition of fat in droplet form) which may lead to cell necroses, inflammation or fibrosis. Fatty degeneration of the liver occurs if the production or intake of fat exceeds the degradation thereof. Fatty degeneration of the liver is present if more than half of liver cells have fatty deposits. It is associated for example with obesity, protein deficiency, diabetes mellitus, chronic alcoholism or as a consequence of necroses after hepatotoxins. Intravenous administration of the medicament Essentiale® can have a beneficial effect on the progress of these liver disorders.

It is reported that fat pads like those occurring under the eyes, on the abdomen or on the hips of overweight people shrink, and there are said to be esthetic improvements in the appearance of the treated people, if these people received subcutaneous injection of Lipostabil® N i.V. (Patricia Guedes Rittes, The Use of Phosphatidylcholine for Correction of Lower Lid Bulging Due to Prominent Fat Pads, Dermatol. Surg. 2001;27: 391-392). Lipostabil® N i.V. is a solution for injection which comprises soybean phospholipids, deoxycholic acid, sodium chloride, sodium hydroxide, DL-alpha-tocopherol, ethanol, benzyl alcohol, ethanol and water.

In the attempt to find effective compounds for nonsurgical removal of subcutaneous accumulations of fat, it has now surprisingly been found that subcutaneous administration of the aqueous preparations, employed according to the invention, of this pharmaceutical form Essentiale® N i.V., which have to date been used only for the treatment of liver disorders, also leads to regression of depot fat in the body. Lipolysis of the adipose tissue occurs, and the zone of adipose tissue regresses.

The invention therefore relates to the use of a preparation comprising
a) at least one phospholipid and/or
b) at least one bile acid and
c) component assisting degradation of fat and
d) water
   for producing a medicament for removing subcutaneous accumulations of fat.

The invention further relates to the use of a preparation comprising
a) at least one phospholipid,
b) at least one bile acid,
c) component assisting degradation of fat and
d) water
   for producing a medicament for removing subcutaneous accumulations of fat.

The invention further relates to the use of a preparation comprising
a) at least one phospholipid, and/or
b) at least one bile acid,
c) component assisting degradation of fat,
d) an anti-inflammatory compound and
e) water
   for producing a medicament for removing subcutaneous accumulations of fat.

The invention further relates to the use of a preparation comprising
a) at least one phospholipid,
b) at least one bile acid,
c) component assisting degradation of fat,
d) an anti-inflammatory compound and
e) water
   for producing a medicament for removing subcutaneous accumulations of fat.

The invention further relates to the use of the preparations for producing a medicament for the treatment of adipose tissue disorders, in particular with local derangement of fat distribution.

The invention further relates to the use of the preparations for producing a medicament for regression of adipose tissue tumors.

The invention further relates to the use of the preparations for producing a medicament for the treatment of derangements of fat distribution of an unwanted nature, which are esthetic or pathological in nature, for example lipedemas, lipomatosis of the abdominal walls, dermatopanniculosis deformans or cellulite.

It is possible through the use according to the invention of the preparations to avoid the abovementioned risks and side effects of surgical treatment. In addition, outpatient treatment is more pleasant and less costly for the patient.

The term “phospholipid” means compounds such as 3-sn-phosphatidylcholine, soya (Phospholipon 90), 3-sn-phosphatidylcholine, hydrogenated soya (Phospholipon 90H), 3-(3sn)-phosphatidyl)glycerol soya (Phospholipon G), dimyristoylphosphatidylglycerol, lysophosphatidylcholine or dipalmitoylphosphatidylglycerol, and physiologically tolerated salts thereof.

The term “bile acid” means compounds such as deoxycholic acid, cholic acid, lithocholic acid, chenodeoxycholic acid, hyodeoxycholic acid, trihydroxycoprostanic acid, ursodeoxycholic acid, taurocholic acid or glycocholic acid dipalmitoylphosphatidylglycerol, and the physiologically tolerated salts thereof.

The term “component assisting degradation of fat” means, for example, vitamins such as riboflavin or carnitine. Riboflavin, which is also referred to as vitamin B₂ or lactoflavin, is an alkali- and light-sensitive vitamin which has a yellowish green fluorescence in solution. Riboflavin acts to assist the degradation of fats, carbohydrates and protein. Riboflavin acts in humans in the form of its active coenzymes FAD and FMN in about 60 hydrogen-donating flavoenzymes.

L-Carnitine is β-hydroxy-g-N-trimethylaminobutyrate. It may occur in two different stereoisomers. Only the L form undertakes important functions in the body. D-Carnitine by contrast is harmful to health. L-Carnitine possesses as carrier protein a) catalytic functions in the transport of activated fatty acids and b) metabolic functions as store of activated acetyl radicals. Biotechnological production by bacteria results in only L-carnitine.

The term “antiinflammatory compound” means compounds such as tocopherol or a non-steroidal antiinflammatory drug such as diclofenac or a corticosteroid such as triamcinolone.

Tocopherol or vitamin E is a representative of a group of seven lipid-soluble vitamins with an antioxidant effect; it is a constituent of all membranes of animal cells. The most important naturally occurring compound with vitamin E activity is alpha-tocopherol.

The term “subcutaneous derangements of fat distribution” means adipose tissues in the body of humans and mammals which occur as genetically related or food-related depot fat in the form of localized fat pads and can be regarded as esthetically disturbing critical zones such as abdomen, buttocks, hips, knee, calves, thighs, upper arm, chin, cheeks. They may also involve dystopic proliferation (benign proliferations of the fat cells such as lipomas).

The term “adipose tissue disorders” means for example the following disorders: Lipomas are adipose tissue tumors, which are benign, slow-growing, usually spherical, possibly pedunculated (=I. pendulum) or even villous (=I. arborescens, for example of the synovial villi) mesenchymal tumors composed of—enlarged—adipose tissue cells, preferentially in a subcutaneous cell tissue, possibly with central ossification (=I. ossificans), becoming mucoid (=I. myxomatodes) or calcifying (=I. petrificans), also with increased connective tissue and capsule formation (=I. fibrosum), neoangiogenesis (=I. teleangiectodes), rarely showing malignant degeneration (=I. sarcomatodes, liposarcoma). They are to be categorized as pathological because they grow and their connective tissue envelope may be painful per se, as well as the compression derived therefrom on blood vessels, which may cause neuralgia.

Dercum's disease, called lipomatosis dolorosa, is a special type of hypertrophic proliferation of adipose tissue, which is located between the dermal fat fascia (Kampa's fat fascia) and the underside of the dermis. Hormonal effects lead to an enhanced water-binding capacity of these fat cells which themselves in turn bring about, through pressure phenomena, lymph tract obstructions in the region of the initial fern-like lymph vessels and with which additional compressive and irritant effects are exerted on the peripheral sensory nerves, so that these patients display an extremely painful sensitivity to touch. Over the course of several years up to decades there is formation of irregular fatty nodules in disseminated locations underneath the dermis, which becomes thinner during the aging process, some of which nodules have painful and highly dysesthetic characteristics.

Madelung's neck (Lanois-Bensaude syndrome) is an adipose tissue inflammation with adipose tissue proliferation in which a dystrophic adipose tissue tumor formation is accompanied by subcutaneous scar-like connective tissue compaction. In such cases, surgical procedures can often be only partially successful, because essential anatomic structures are involved in this process and the disorder is manifested essentially in the region of the head, neck and shoulders.

Lipedema is a painful adipose tissue swelling which occurs especially on the lower legs of women and shows a progressive course and characteristics with increasing age.

Piezogenic nodules are nodules on the edges of the hands and the heels which are caused by pressure and occur as multiple adipose tissue hernias, mainly in the medial region of the heel in obese people. They are usually defects in the septation of the subcutaneous adipose tissue which are regarded by patients as cosmetically or functionally disturbing.

Xanthelasma is a pale yellow, slightly raised plaque-like deposit of cholesterol in the region of the eyelids. They are soft and easily displaceable and usually occur symmetrically on both eyes. It is caused by local derangements of lipid metabolism. Postmenopausal women are affected particularly frequently. Diabetes mellitus and elevated blood lipid levels are also associated with an increased risk of developing it. Xanthelasmas may cause psychological stress because of their appearance.

The term “regression” means the lipolysis of the adipose tissue and regression of the proliferated adipose region.

The abovementioned adipose tissue disorders show, in contrast to the food-related lipohypertrophy (which is also followed by a deposition of fat in the sense of the derangement of fat distribution), tissue conditions or entities which can be pathologically differentiated unambiguously and which can be described by histological parameters of scarring and inflammation, but also by connective tissue encapsulations and by changes in the histological adipose tissue morphology itself.

The invention further relates to the use of preparations for producing a medicament for the treatment of cellulite.

Cellulite is a special type of hypertrophic proliferation of adipose tissue, which is located between the dermal fat fascia (Kampa's fat fascia) and the underside of the dermis. Hormonal effects lead to an enhanced water-binding capacity of these fat cells which themselves in turn bring about, through pressure phenomena, lymph tract obstructions in the region of the initial fern-like lymph vessels. Over the course of several years up to decades there is formation of irregular fatty nodules in disseminated locations underneath the dermis, which becomes thinner during the aging process, some of which nodules have painful and highly dysesthetic characteristics.

The invention relates in particular in the claimed pharmaceutical forms to the use of phospholipid in which the phospholipid is in the form of a physiologically tolerated salt, for example as sodium, potassium and/or ammonium salt.

The phospholipid can be isolated from oil seeds, rapeseed, soybean or sunflowers and, after appropriate application, be employed in the liposome system. Lecithin, for example from chicken egg, is also suitable. Phospholipids from soybeans are preferred.

The invention also relates to the use of phospholipid in which the phospholipid is the phosphatidylcholine from soybean and is isolated therefrom. Especially when the phospholipid consists of at least 90 percent by weight (% by weight) of soybean phosphatidylcholine, in particular 95% by weight.

The invention also relates to the use of a bile acid or different bile acids, in which the bile acid is in the form of a physiologically tolerated salt. This may be for example a sodium, potassium and/or ammonium salt of deoxycholic acid, cholic acid, lithocholic acid, chenodeoxycholic acid, hypodeoxycholic acid, trihydroxycoprostanic acid, ursodeoxycholic acid, taurocholic acid or glycocholic acid.

The mass ratio of phospholipid to bile acid is, in % by weight, from 30:1 to 1:0.03, preferably from 1:0.7 to 1:0.1, in particular 1:0.6 to 1:0.3.

The phospholipid concentration in the liposome system is from 0.5% by weight to 30% by weight, preferably from 5% by weight to 25% by weight, in particular from 10% by weight to 20% by weight.

The liposomes have a diameter of from 30 nm to 180 nm, preferably from 30 nm to 130 nm, in particular from 50 nm to 90 nm. These liposomes can be sterilized by filtration without difficulty, employing filters with a pore diameter of 0.2 μm.

The pH of the medicament is in the range from 6.5 to 9.0, preferably from 6.5 to 8.0, in particular from 6.5 to 7.4.

The weight ratio of the component assisting degradation of fat in the preparation is from 0.00001 percent by weight to 20 percent by weight, preferably from 0.0001% by weight to 10% by weight, in particular from 0.001% by weight to 1% by weight.

The weight ratio of the antiinflammatory compound in the preparation depends on the nature of the antiinflammatory compound and is ordinarily from 0.00001 to 20 percent by weight.

The preparations of the invention are produced, for example, by dissolving or dispersing at least one phospholipid and/or at least one bile acid in the abovementioned ratio to one another in an organic solvent, and then adding the components assisting the degradation of fat. It is possible where appropriate then to add an antiinflammatory compound.

This solution or dispersion is subsequently concentrated, and then water is added. Production of the preparations of the invention can after addition of the water be promoted by extrusion, high-pressure homogenization and/or ultrasound treatment.

The treatment takes place below 40° C., preferably from 20° C. to 30° C. Suitable organic solvents are ethanol, propanol, isopropyl alcohol or benzyl alcohol, each alone or in a mixture. The residual volumes of alcohol after concentration should be from 0 percent by volume (vol. %) to 20 vol. %, preferably from 0 vol. % to 10 vol. %.

Processes for producing the preparations are also described in European patent applications EP 0 470 437 or EP 0 615 746.

It is possible where appropriate to add to the preparations of the invention also antioxidants such as ascorbic acid, sodium bisulfite or sodium pyrosulfite, or preservatives such as benzyl alcohol.

The preparations may also comprise colloidal structures such as micelles or mixed micelles. These structures have a particle diameter of from 1 to 50 nm. They consist of bile acid and phospholipid. The mass ratio of bile acid to phospholipid is in % by weight from 0.1:2 to 2:1, preferably from 1:2. The phospholipid concentration in the colloidal structures in the medicaments is from 5% by weight to 15% by weight, preferably from 10% by weight. The colloidal structures are produced for example by dissolving the bile acid in water, making the solution somewhat alkaline. The phospholipid is then dispersed therein. The component assisting the degradation of fat is then added and, where appropriate, an antiinflammatory compound can then be added. Filtration is finally carried out.

The preparation employed according to the invention, and comparable pharmaceutical forms, are administered by subcutaneous, intra-articular, intraperitoneal, intramuscular injection or short infusions. Subcutaneous injection or infusion is preferred. On application to large areas, administration of Essentiale by means of the tumenescence technique is to be regarded as a particularly suitable method. This entails in the first step up to 8 liters of a saline solution including anesthetics and substances having antiinflammatory activity being infiltrated into the adipose tissue, and the adipose tissue being mobilized. The main mass of the fat is then sucked out. Addition of Essentiale to the infiltrate assists liposuction by medicinal lipolysis. The infiltration method allows particularly good exposure of Essentiale in the target tissue.

Percutaneous administration is also claimed, in various carrier media and with use of various aids, for example iontophoresis.

Simultaneous introduction of the preparations and pharmaceutical forms employed according to the invention can also take place in particular applications via a tumescence method which makes use of the hydrostatic pressure in order to ensure uniform distribution.

Percutaneous administration is also possible, which can take place in various carrier media such as creams, ointments, gels, hydrogels, lotions or pastes, and with use of various aids, for example, iontophoresis or phonophoresis.

Suitable preparations and pharmaceutical forms are, for example, suspensions, emulsions or injectable solutions, and products with protracted release of active ingredient, in the production of which conventional aids such as are used. The preparations can also be in the form of a concentrate, dry substance or lyophilizates, in order to increase the stability for example.

These pharmaceutical products are preferably produced and administered in dosage units, each unit comprising a particular dose of the preparation as active ingredient. In the case of solutions for injection in ampoule form, this dose can be from about 10 mg to about 2000 mg, preferably from about 50 mg to about 2000 mg, with preference from about 250 mg to 500 mg, based on the phospholipid.

Daily doses required for the treatment of an adult patient are, depending on the size of the treated adipose tissue, on administration of solutions for injection from 5 mg to 500 mg, preferably 250 mg to 500 mg, per injection, based on the phospholipid. The solutions for injection can also be diluted before administration, preferably with saline solution. However, in some circumstances, higher or lower daily doses may also be appropriate. The dose also depends on the size of the lipomas, and for small lipomas amounts of from 1 mg to 50 mg, preferably 2 mg to 20 mg, per injection, based on the phospholipid, are entirely sufficient. Administration of the daily dose can take place both through a single dose in the form of a single dosage unit or else a plurality of small dosage units and by multiple dosage of divided doses at defined intervals.

The invention is explained in more detail by means of examples below.

### EXAMPLE 1

### Treatment of Lipohypertrophy with the Aid of Intralesional Injection of Essentiale® N i.V.

A female patient 48 years old with periumbilical adipose tissue proliferation still had a residual layer of 3.11 cm of fat after liposuction on two previous occasions. The patient underwent two injection of Essentiale® N i.V. (Rote Liste, March 2003; ingredients: soybean phospholipids, comprising 93% (3-sn-phosphatidyl)choline (extractant 95% (v/v) ethanol 250 mg, deoxycholic acid, sodium chloride, sodium hydroxide, riboflavin, D,L-alpha-tocopherol, ethanol, water for injections, as preservative 45 mg of benzyl alcohol) at an interval of 4 weeks. Injection took place into the subcutaneous adipose tissue with in each case 30 ml of a preparation of Essentiale® N i.V. diluted by 50% with physiological saline solution. After 8 weeks it was possible to detect a reduction in the adipose tissue thickness to 55% (adipose tissue thickness 1.41 cm) of the original thickness. The treated correlating skin surface zone umbilically amounted to 25 cm*15 cm. The follow-up period now free of recurrence amounted to 6 months.

1. A method for removing subcutaneous accumulations of fat comprising the administration of an efficacious amount of a preparation comprising
a) at least one phospholipid or
b) at least one bile acid,
c) a component assisting degradation of fat and
d) water.

2. The method of claim 1, wherein the preparation comprises
a) at least one phospholipid,
b) at least one bile acid,
c) a component assisting degradation of fat and
d) water

3. The method of claim 1, wherein the preparation further comprises an antiinflammatory compound.

4. The method of claim 2, wherein the preparation further comprises an antiinflammatory compound.

5. A method for the treatment of adipose tissue disorders which are local derangements of fat distribution, the method comprising the removing of subcutaneous accumulations of fat in accordance with the method of claim 1.

6. A method for the regression of adipose tissue tumors comprising the removing of subcutaneous accumulations of fat in accordance with the method of claim 1.

7. The method of claim 5, wherein the local derangements of fat distribution are of an unwanted esthetic or pathological nature, and are lipedemas, lipomatosis of the abdominal walls, dermatopanniculosis deformans, xanthelasma, piezogenic modules or cellulite.

8. The method of claim 1, wherein the phospholipid employed is one of the following compounds 3-sn-phosphatidylcholine, soya (Phospholipon 90), 3-sn-phosphatidylcholine, hydrogenated soya (Phospholipon 90H), 3-(3sn)-phosphatidyl)glycerol soya (Phospholipon G), dimyristoylphosphatidylglycerol, lysophosphatidylcholine or dipalmitoylphosphatidylglycerol, and physiologically tolerated salts thereof, or a mixture of these compounds.

9. The method of claim 8, wherein the physiologically tolerated salt of the phospholipid employed is its sodium, potassium or ammonium salt.

10. The method of claim 8, wherein soybean phosphatidylcholine is employed as the phospholipid.

11. The method of claim 10, wherein the phospholipid consists of at least 90% by weight soybean phosphatidylcholine.

12. The method of claim 1, wherein the bile acid employed is selected from the group consisting of deoxycholic acid, cholic acid, lithocholic acid, chenodeoxycholic acid, hyodeoxycholic acid, trihydroxycoprostanic acid, ursodeoxycholic acid, taurocholic acid and glycocholic acid, and the physiologically tolerated salts thereof, or a mixture thereof.

13. The method of claim 12, wherein the physiologically tolerated salt of the bile acid employed is its sodium, potassium or ammonium salt.

14. The method of claim 2, wherein the mass ratio of phospholipid to the bile acid in percent by weight is from 30:1 to 1:0.03.

15. The method of claim 1, wherein the phospholipid concentration is from 0.5% by weight to 30% by weight in the preparation.

16. The method of claim 1, wherein the component assisting degradation of fat is riboflavin or carnitine or a mixture of these components.

17. The method of claim 3, wherein the antiinflammatory compound is tocopherol, diclofenac or triamcinolone or a mixture of these compounds.

18. The method of claim 1, wherein the amount of the component assisting degradation of fat in the preparation is from 0.00001 percent by weight to 20 percent by weight.

19. The method of claim 1, wherein the amount of the antiinflammatory compound in the preparation is from 0.00001 to 20 percent by weight.

20. The method of claim 1, wherein the preparation is administered by subcutaneous, intra-articular, intraperitoneal, intramuscular injection, short infusions or infusion, or by use of the tumenescence technique.
